(19) United States
(12) Patent Application Publication
(10) Pub. No.: US 2023/0381419 A1
(43) Pub. Date: Nov. 30, 2023

(54) Medical Injection Device Having a Biasing Component Engageable with a Plunger Rod

(71) Applicant: Becton, Dickinson and Company, Franklin Lakes, NJ (US)

(72) Inventors: Stephen C. Simmons, Ringwood, NJ (US); Evan Leibowitz, Linden, NJ (US); David Rothery, Melksham (GB); Stephen Simantiras, Randolph, NJ (US)

(21) Appl. No.: 18/028,355

(22) PCT Filed: Sep. 23, 2021
(86) PCT No.: PCT/US2021/051702

§ 371 Date: Mar. 31, 2023

Related U.S. Application Data
(60) Provisional application No. 63/083,588, filed on Sep. 25, 2020.

(51) Int. Cl. A61M 5/315

(52) U.S. Cl. CPC A61M 5/31511; A61M 5/31536

(57) ABSTRACT

A medical injection device including a housing having a distal end and a proximal end, a brake tower having a proximal end portion, a distal end portion, and a sidewall therebetween defining a longitudinal axis, and a cartridge received within the housing distally of the brake tower and holding a composition therein. The device further a piston rod coaxially positioned within the brake tower and capable of axial travel within the brake tower and the cartridge along the longitudinal axis. A biasing component is positioned between the proximal end portion of the cartridge and the distal end portion of the brake tower, where the biasing component surrounds a portion of the piston rod.

### CROSS-REFERENCE TO RELATED APPLICATION

The present application claims priority to U.S. Provisional Application Ser. No. 63/083,588, entitled “Medical Injection Device Having a Biasing Component Engageable with a Plunger Rod”, filed Sep. 25, 2020, the entire disclosure of which is hereby incorporated by reference in its entirety.

### BACKGROUND OF THE INVENTION

### Field of the Invention

The present disclosure relates generally to components useful as replacements for helical coil springs for medical devices, and, in particular embodiments or aspects, to injection devices including a biasing component such as, e.g., a wave leaf spring engageable with a cartridge, a brake tower, and a plunger rod of the injection device.

### Description of Related Art

Medical injection devices often utilize resilient members, such as springs, placed between various components, to bias the components in order to ensure more accurate drug delivery. However, there are shortcomings to the use of current resilient members used in medical injection devices, such as a lack of control and load recovery. Accordingly, there is a need in the art for a more robust component to allow for greater control and load recovery.

### SUMMARY OF THE INVENTION

Provided herein is a medical injection device including a housing having a distal end and a proximal end, a brake tower having a proximal end portion, a distal end portion, and a sidewall therebetween defining a longitudinal axis, a cartridge received within the housing distally of the brake tower and holding a composition therein, where a proximal end portion of the cartridge is configured to extend at least partially within the distal end portion of the brake tower along the longitudinal axis, a piston rod coaxially positioned within the brake tower and capable of axial travel within the brake tower and the cartridge along the longitudinal axis, and a biasing component positioned between the proximal end portion of the cartridge and the distal end portion of the brake tower, where the biasing component surrounds a portion of the piston rod.

The biasing component may be configured to engage an outer surface of the piston rod. The biasing component may include a plurality of inwardly-projecting protrusions. The plurality of inwardly-projecting protrusions of the biasing component may be configured to engage an outer surface of the piston rod. The plurality of inwardly-projecting protrusions may be configured to engage the outer surface of the piston rod when the biasing component is in a substantially uncompressed configuration, where the plurality of inwardly projecting protrusions are configured to disengage from the outer surface of the piston rod when the biasing component is in a substantially compressed configuration. Each of the inwardly-projecting protrusions may include a prong configured to engage the piston rod. The plurality of inwardly-projecting protrusions may include four protrusions. The biasing component may include a plurality of notches formed therein, where the plurality of notches are configured to allow for flexing of engagement surfaces of the biasing component. The biasing component may be configured as a leaf spring. The biasing component may be formed from metal. The biasing component may be formed from polymer.

In a further aspect or embodiment, a medical injection device includes a housing having a distal end and a proximal end, a brake tower having a proximal end portion, a distal end portion, and a sidewall therebetween defining a longitudinal axis, a cartridge received within the housing distally of the brake tower and holding a composition therein, where a proximal end portion of the cartridge is configured to extend at least partially within the distal end portion of the brake tower along the longitudinal axis, a cartridge housing received within the housing distally of the brake member and configured to hold the cartridge therein, an injection needle at a distal end of the housing and in fluid communication with the cartridge, an actuation member at a proximal end of the housing, the actuation member configured to actuate the medical injection device to deliver the composition through the injection needle, a piston rod coaxially positioned within the brake tower and capable of axial travel within the brake tower and the cartridge along the longitudinal axis, and a biasing component positioned between the proximal end portion of the cartridge and the distal end portion of the brake tower, where the biasing component comprises a plurality of inwardly-projecting protrusions configured to engage an outer surface of the piston rod.

The plurality of inwardly-projecting protrusions may be configured to engage the outer surface of the piston rod when the biasing component is in a substantially uncompressed configuration, where the plurality of inwardly projecting protrusions are configured to disengage from the outer surface of the piston rod when the biasing component is in a substantially compressed configuration. Each of the inwardly-projecting protrusions may include a prong configured to engage the outer surface of the piston rod. The plurality of inwardly-projecting protrusions may include four protrusions. The biasing component may be formed from metal. The biasing component may be formed from polymer.

In a further aspect or embodiment, a medical injection device includes a housing having a distal end and a proximal end, a brake tower having a proximal end portion, a distal end portion, and a sidewall therebetween defining a longitudinal axis, a cartridge received within the housing distally of the brake tower and holding a composition therein, where a proximal end portion of the cartridge is configured to extend at least partially within the distal end portion of the brake tower along the longitudinal axis, a cartridge housing received within the housing distally of the brake member and configured to hold the cartridge therein, an injection needle at a distal end of the housing and in fluid communication with the cartridge, an actuation member at a proximal end of the housing, the actuation member configured to actuate the medical injection device to deliver the composition through the injection needle, a piston rod coaxially positioned within the brake tower and capable of axial travel within the brake tower and the cartridge along the longitudinal axis, and a biasing component positioned between the proximal end portion of the cartridge and the distal end portion of the brake tower, where the biasing component comprises a plurality of notches configured to allow for flexing of engagement surfaces of the biasing component.

The biasing component may be configured as a leaf spring. The biasing component may be configured to engage an outer surface of the piston rod.

### BRIEF DESCRIPTION OF THE DRAWINGS

FIG. 1 is an exploded view of a medical injection device with a biasing component engageable with a plunger rod according to a non-limiting embodiment or aspect as described herein;

FIG. 2 is a cross-sectional view of a medical injection device including a biasing component engageable with a plunger rod according to a non-limiting embodiment or aspect as described herein;

FIG. 3 is a perspective view of a biasing component engaged with a plunger rod according to a non-limiting embodiment or aspect as described herein;

FIG. 4 is a partial cross-sectional view of a medical injection device with a brake tower, a plunger rod, and a biasing component according to a non-limiting embodiment or aspect as described herein, showing a pre-engagement position;

FIG. 5 is a partial cross-sectional view of a medical injection device with a brake tower, a plunger rod, a cartridge, and a biasing component according to a non-limiting embodiment or aspect as described herein, showing an engagement position;

FIG. 6 a top view of a biasing component engaged with a plunger rod according to another non-limiting embodiment or aspect as described herein;

FIG. 7 is a perspective view of a biasing component engaged with a plunger rod according to another non-limiting embodiment or aspect as described herein;

FIG. 8 is a partial cross-sectional view of a medical injection device with a brake tower, a plunger rod, and a biasing component according to a non-limiting embodiment or aspect as described herein, showing a pre-engagement position; and

FIG. 9 is a partial cross-sectional view of a medical injection device with a brake tower, a plunger rod, a cartridge, and a biasing component according to a non-limiting embodiment or aspect as described herein, showing an engagement position.

### DESCRIPTION OF THE INVENTION

The use of numerical values in the various ranges specified in this application, unless expressly indicated otherwise, are stated as approximations as though the minimum and maximum values within the stated ranges are both preceded by the word “about”. As used herein, the term “about” means the stated value ±10%. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases.

The figures accompanying this application are representative in nature, and should not be construed as implying any particular scale or directionality, unless otherwise indicated. For purposes of the description hereinafter, the terms “upper”, “lower”, “right”, “left”, “vertical”, “horizontal”, “top”, “bottom”, “lateral”, “longitudinal” and derivatives thereof shall relate to the invention as it is oriented in the drawing figures. However, it is to be understood that the invention may assume various alternative variations and step sequences, except where expressly specified to the contrary. Hence, specific dimensions and other physical characteristics related to the embodiments disclosed herein are not to be considered as limiting.

Provided herein is a brake tower with a biasing component for use as a replacement for a spring in a medical device.

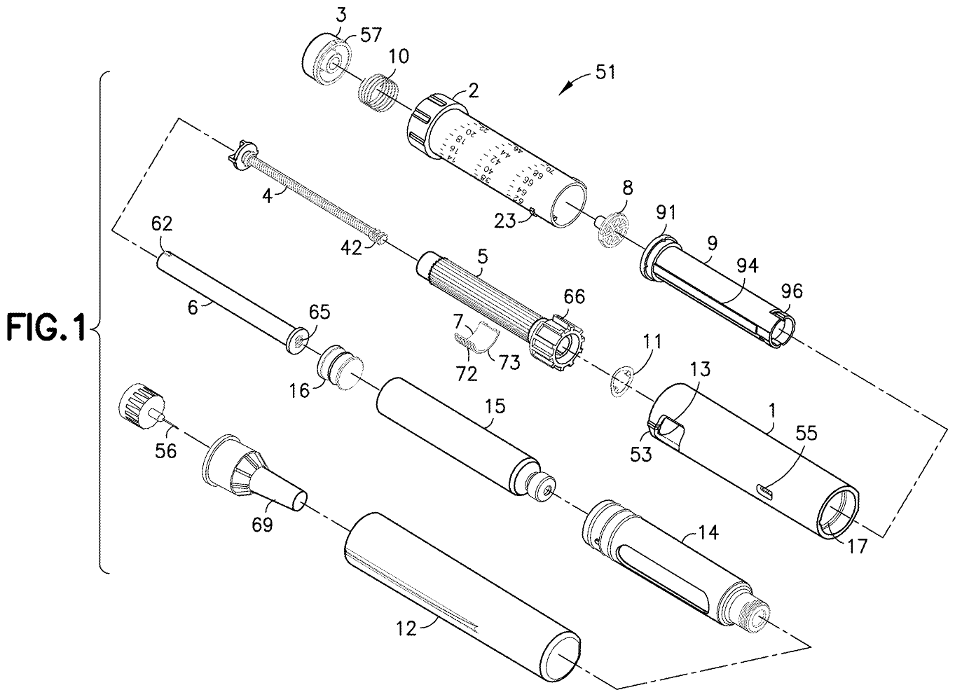

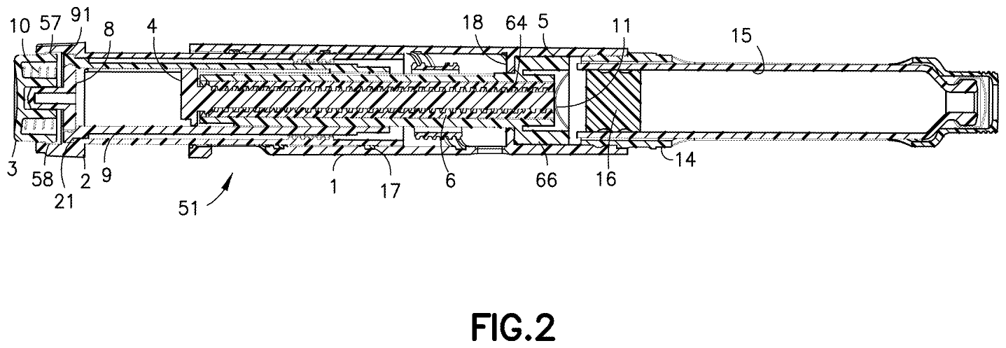

Referring to FIGS. 1 and 2, a medical injection device in accordance with an aspect of the present disclosure is shown. Such devices, such as injection pens, are described in U.S. Pat. No. 9,421,334, which is incorporated herein by reference in its entirety. With reference to FIGS. 1 and 2, shown are an exploded (FIG. 1) and cross-sectional (FIG. 2) view of an injection pen 51 for delivery of a composition to a user. As shown, injection pen 51 includes a pen upper body or housing 1, which houses a plurality of dose setting and injection components. Pen upper body 1 is connected to a cartridge housing 14, which houses cartridge 15. Injection pen 51 can also include a lower pen cap 12 to cover the cartridge 15 and cartridge housing 14 when injection pen 51 is not in use. As shown, injection pen 51 can include a dose set knob 2 that includes a knob-like portion that is rotated by a user to set a desired dose. Dose set knob 2 can also include a plurality of numerals, corresponding to a number of dosage units that is visible through a window 13 provided on pen upper body 1. A user rotates dose set knob 2 until the desired dose is visible in window 13. Pen upper body 1 can include an arrow or other indicator 53 to precisely indicate the set dose. Once the desired dose is set, a user presses a button 3 until the set dosage amount is completely injected. An outer shield 69 can cover a needle 56 to prevent accidental needle sticks upon removal of the lower pen cap 12.

Injection pen 51 can include push button 3, provided at a proximal end, closest to a user and farthest from needle 56, of pen upper body 1. Push button 3 can include an annular bead or rim 57 that engages with a corresponding annular groove (not shown) provided on the internal surface of dose set knob 2. The annular rim and groove connection can be a friction fit that maintains push button 3 in a biased position on dose set knob 2 under the force of a button spring 10, but allows push button 3 to be pushed into dose set knob 2 for injecting a set dose. The interior of push button 3 can accommodate a setback bearing insert 8 that rests on an internal surface at a proximal end of a setback member or driver 9. Push button 3 can be designed to rotate freely on setback bearing insert 8.

Setback member or driver 9 can be a cylindrical member, coaxial with and surrounded by dose set knob 2. Setback member 9 can be provided co-axially around a brake tower 5. Brake tower 5 can be axially and rotatably fixed to pen upper body 1. In non-limiting embodiments or aspects, brake tower 5 co-axially surrounds a piston rod 6. Piston rod 6 can include a set of keys (not shown) that engage a slot (not shown) internal to brake tower 5 to rotatably lock piston rod 6 to brake tower 5. Piston rod 6 can include a plurality of threads (not shown) provided on the interior surface thereof. Piston rod 6 can co-axially surround a lead screw 4 that includes a series of threads 42 at least at its distal end. Lead screw threads 42 can be configured to be in threaded engagement with the internal threads (not shown) provided on the interior of piston rod 6. Due to its threaded engagement with lead screw 4, piston rod 6 can be moved into cartridge 15 during injection to press on a stopper 16 provided inside cartridge 15 to expel a dose of medication.

Following assembly, and when injection pen 51 is ready to be used, a dose for drug delivery can be set. To set a dose using injection pen 51 as described herein, a user rotates the knob portion of dose set knob 2 relative to pen upper body 1. An outer surface 59 of the dose set knob 2 can include a thread 23, which is in threaded engagement with a plurality of threads 17 provided on the internal surface of the pen upper body 1, as shown in FIGS. 1 and 2. Accordingly, as dose set knob 2 is rotated relative to pen upper body 1, dose set knob 2 screws or advances a distance out of pen upper body 1, as shown in FIG. 2. Dose set knob 2 can include an annular shoulder or rim 21 on the interior surface thereof near the proximal end. This annular shoulder 21 can engage with an enlarged portion or head 91 of setback member 9, as shown in FIG. 2. The annular shoulder 21 of dose set knob 2 can include a series of teeth or ridges that engage with a plurality of similarly shaped teeth or ridges (not shown) provided on enlarged head 91 of setback member 9. Dose set knob teeth and setback member teeth can extend in opposite axial directions. During dose setting, dose set knob 2 can be free to rotate with respect to setback member 9 in both clockwise and counter-clockwise directions. As this occurs, the plurality of teeth or ridges on dose set knob 2 can slip past the teeth provided on head portion 91 of setback member 9, thus providing a tactile signal or clicking noise to indicate the setting of a dosage amount.

With regard to the injection mechanism, as described above, lead screw 4 can include a plurality of threads 42 at its distal end that are in threaded engagement with a plurality of threads 64 that can be provided along the entire length of a hollow piston rod 6 as shown in FIG. 1. Piston rod 6 can be held non-rotatably with respect to pen upper body 1 due to a non-rotatable coupling with brake tower 5, which can be held axially and rotatably fixed with respect to the pen upper body 1. Piston rod 6 can include a key or set of keys 62 at its distal end that engage with a slot (not shown) provided on the internal surface of the brake tower 5 to prevent relative rotation therebetween while permitting piston rod 6 to move axially with respect thereto. Threads 42 of lead screw 4 can include a flat portion (not shown) corresponding to a flat portion 65 of piston rod 6, such that axial movement of the lead screw during dose setting does not result in axial movement of the piston rod 6. Accordingly, rotation of lead screw 4 during injection of a dose can cause the threads 42 of lead screw 4 to engage threads 64 of the piston rod 6, thereby axially moving piston rod 6. Because piston rod 6 can be non-rotatable with respect to body 1, as lead screw 4 is caused to rotate during injection, as described above due to its rotational coupling setback member 9, piston rod 6 through its threaded engagement with lead screw 4 can be caused to move in the distal direction to press against stopper 16 provided in cartridge 15, thus expelling a liquid medication therefrom.

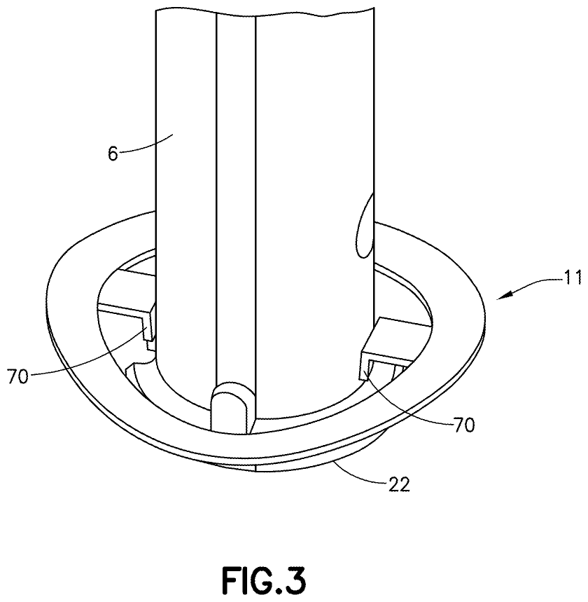

With reference to the present disclosure, injection pen 51 includes brake tower 5 and cartridge 15, as described above. In previous injections pens, such as that which is described in U.S. Pat. No. 9,421,334, a wave clip (or wave spring) has been provided between a distal end of the brake tower 5 and the cartridge 15 so as to bias the cartridge 15 in a distal direction to prevent any movement of the cartridge 15 during injection, thereby ensuring that an accurate dose is injected. However, the use of a wave clip alone has shortcomings, such as, e.g., lack of control and load recovery. Thus, in accordance with an aspect of the present disclosure, and as is shown in FIGS. 1 and 2, injection pen 51 includes both a biasing component 11 configured to achieve the biasing of cartridge 15 with respect to brake tower 5. As will be described in further detail below with respect to FIGS. 3-6, the biasing component 11 is engageable with the piston rod 6 so as to hold the biasing component 11 in place relative to the brake tower 5, and once compressed and restrained by the brake tower 5 and cartridge 15, allows the plunger rod 6 unrestricted travel.

Referring to FIG. 3, shown is a non-limiting embodiment of the biasing component 11 for a medical injection device. The biasing component 11 may be any appropriate resilient member such as, e.g., a sinusoidal (or “wave”) spring washer, leaf spring, or Belleville spring, etc. The biasing component 11 may be formed of any appropriate material such as, e.g., a polymeric material, a thermoplastic, a metal or metal alloy, etc. In one aspect or embodiment, the biasing component 11 is formed from stainless steel. In one aspect or embodiment, a spring constant is selected to ensure that the weight of the cartridge 15 and any flexure of the cartridge septum is accommodated while maintaining the required biasing force. In one aspect or embodiment, the biasing component 11 is provided in a range of different heights with a height of the biasing component 11 being selected based on a length of the cartridge 15.

As shown in FIG. 3, the biasing component 11 is configured to be engageable with the piston rod 6 of the injection pen 51 at or near a distal end portion of the piston rod 6. Specifically, the biasing component 11 includes a plurality of inwardly-projecting protrusions 70 configured to selectively engage an outer sidewall of the piston rod 6 or piston flange 22. In a non-limiting embodiment or aspect, the protrusions 70 may include prongs or other engagement surfaces that allow the biasing component 11 to rest on the piston rod flange 22 yet have clearance to allow free travel along the length of the piston sidewall. Due to the presence of a flange 22 on piston rod 6, the biasing component 11 is prevented from full disengagement relative to the piston rod 6. Thus, unlike previous biasing components used in injection pens, the biasing component 11 in accordance with aspects of the present disclosure is “free floating” though constrained within the medical device.

In one aspect or embodiment, when the biasing component 11 is in a substantially uncompressed state, the protrusions 70 are configured to grip the outer diameter of the plunger rod 6, thereby retaining the biasing component 11 relative to the plunger rod 6. However, when the biasing component 11 is compressed (i.e., flattened), the protrusions 70 are configured to pull away from the outer diameter of the plunger rod 6, thereby releasing the engagement between biasing component 11 and the plunger rod 6. In this way, when the biasing component 11 is at least partially compressed, the plunger rod 6 is capable of axial movement without contact from the protrusions 70 and, thus, without corresponding axial movement of the biasing component 11.

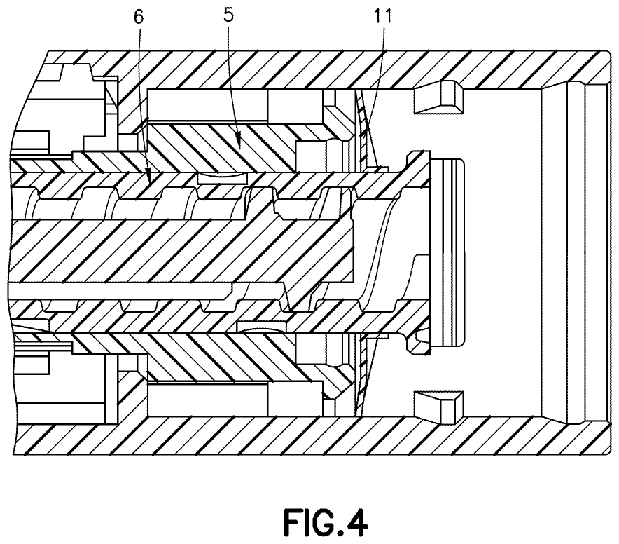

Referring to FIGS. 4-6, a non-limiting embodiment or aspect of an interface between a brake tower 5 and cartridge 15 for a medical injection device is shown. A brake tower can assume any useful configuration for use in a medical injection device. In non-limiting embodiments or aspects, brake tower 5 is cylindrical, and has a proximal end, a distal end, and a sidewall therebetween defining a longitudinal axis. In non-limiting embodiments or aspects, brake tower 5 is at least partially hollow, and the proximal end, distal end, and sidewall define an at least partially open interior that can receive one or more other components of a medical injection device. For example, the piston rod 6 may extend at least partially through the interior of the brake tower 5.

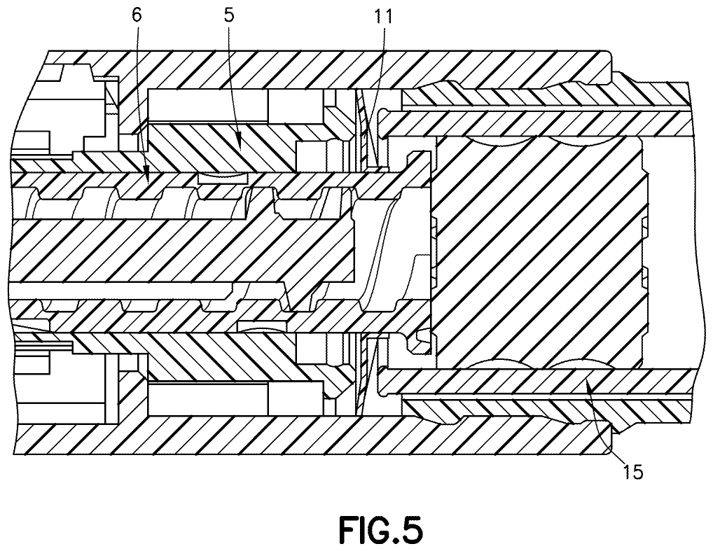

In non-limiting embodiments or aspects, the distal end portion of brake tower 5 is enlarged, e.g., by having a greater circumference than the proximal end. In non-limiting embodiments or aspects, distal end of brake tower 5 is configured to partially receive a proximal end of cartridge 15, as is shown in FIG. 5. As described above, the biasing component 11 is positioned in engagement with the piston rod 6 and within the interior of the distal end of the brake tower 5 between the proximal end of the cartridge 15 and an interior shoulder surface the brake tower 5 so as to bias the cartridge 15 in a distal direction to prevent any movement of the cartridge 15 during injection. Furthermore, as was also described above, the engagement of the biasing component 11 with both the brake tower 5 and the cartridge 15 allows the piston rod 6 to move axially relative to the brake tower 5 and the cartridge 15 due to the clearance with the piston rod side wall provided on the biasing component 11 or the release of the protrusions 70 from engagement with the outer diameter of the piston rod 6.

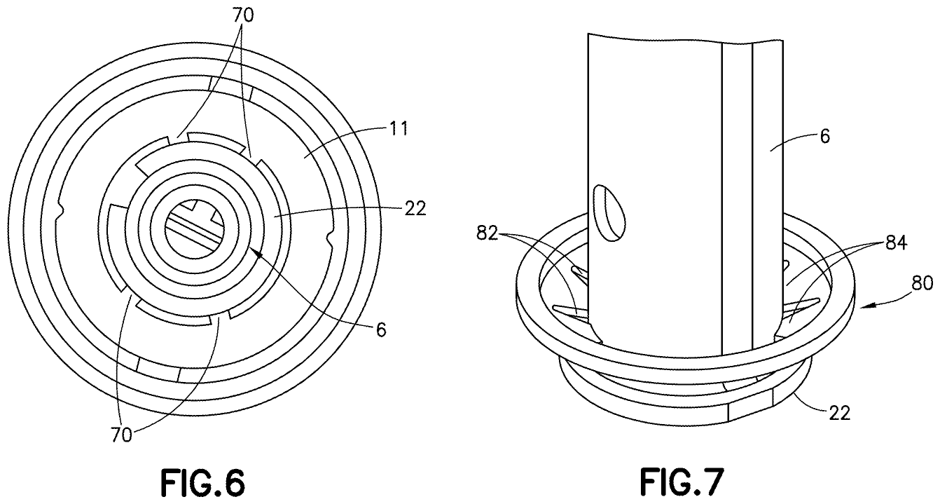

Referring to FIG. 6, the biasing component 11 is shown as having a total of four protrusions 70, which are each capable of engaging the outer diameter of piston rod 6 in certain configurations. However, it is to be understood that more or fewer protrusions 70 may be utilized in accordance with other non-limiting embodiments or aspects of the present disclosure. In one aspect or embodiment, two protrusions 70 are provided, although any number of protrusions 70 may be placed on the sections of the biasing component 11 which deflect outward as the spring is flattened.

Next, referring to FIGS. 7-9, and in accordance with another non-limiting embodiment or aspect of the present disclosure, a biasing component 80 for use in a medical injection device is shown.

Biasing component 80 may be any appropriate component (e.g., a Belleville spring, etc.) that surrounds the outer diameter of the piston rod 6 of the injection device, as shown in FIG. 7. As with biasing component 11 described above, biasing component 80 is capable of selectively engaging with and gripping the outer diameter or flange surface of a piston rod 6 of the injection device.

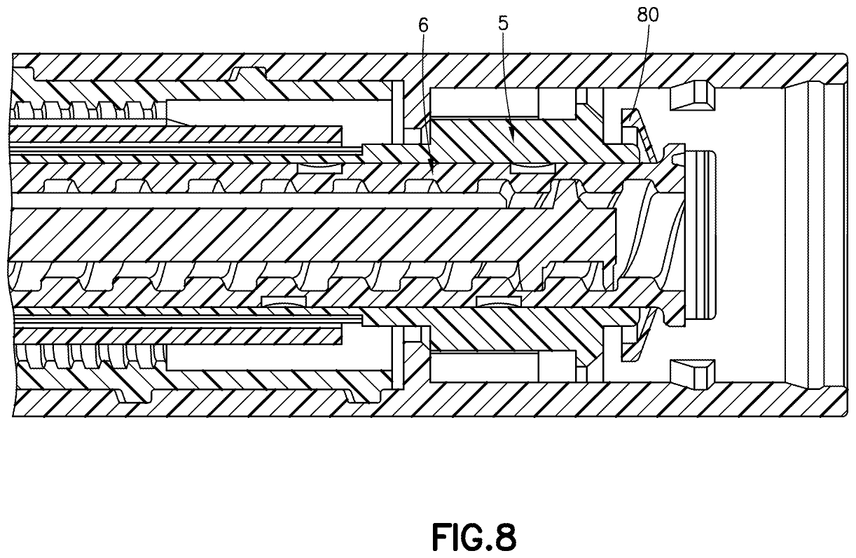

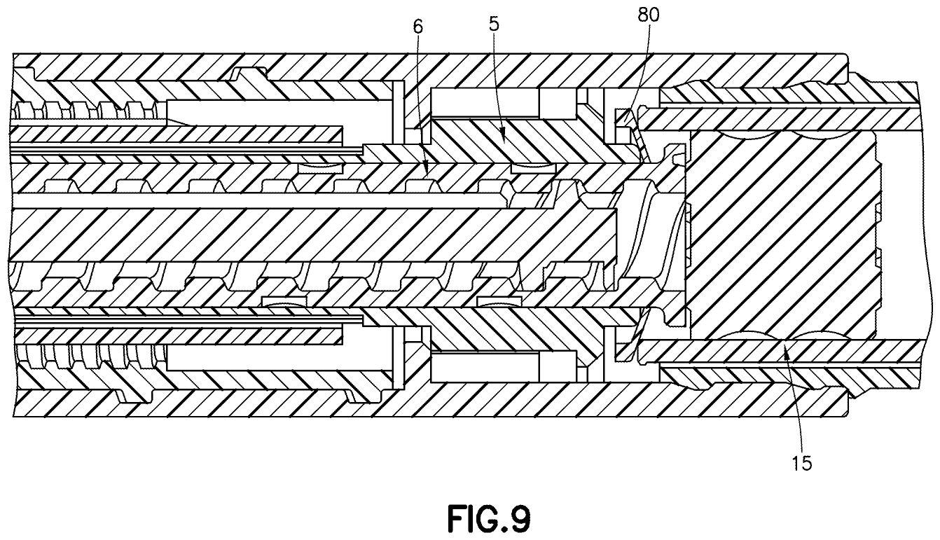

As shown in FIG. 7, the biasing component 80 may include notches 82 in the material, which allows for flexing of respective engagement surfaces 84. Such a configuration enables piston rod 6 to move freely through the inner diameter of the biasing component 80, while still retaining biasing component 80 on the piston rod 6 or flange. As shown in FIGS. 8 and 9, prior to assembly and insertion of the cartridge 15, the biasing component 80 is engaged with the piston rod diameter or flange 6 (FIG. 8) and, after insertion of the cartridge 15, the biasing component 80 is biased when engaged with the brake tower 5 and the cartridge 15.

As described above, the biasing component 80 may be any appropriate resilient member such as, e.g., a sinusoidal (or “wave”) spring washer, star washer, Belleville spring or E-clip. However, it is to be understood that other types of biasing components and/or springs may be utilized. Furthermore, the biasing component 80 may be formed of any appropriate material such as, e.g., an elastomer such as silicone, a polymeric material, a thermoplastic, a metal or metal alloy, etc. In one aspect or embodiment, the biasing component 80 is formed from stainless steel.

Although the devices have been described in detail for the purpose of illustration based on what is currently considered to be the most practical and preferred embodiments, it is to be understood that such detail is solely for that purpose and that the systems and methods are not limited to the disclosed embodiments, but on the contrary, is intended to cover modifications and equivalent arrangements that are within the spirit and scope of the appended claims. For example, it is to be understood that the present systems and methods contemplate that, to the extent possible, one or more features of any embodiment can be combined with one or more features of any other embodiment.

The invention claimed is:

1. A medical injection device comprising:
a housing having a distal end and a proximal end;
a brake tower having a proximal end portion, a distal end portion, and a sidewall therebetween defining a longitudinal axis;
a cartridge received within the housing distally of the brake tower and holding a composition therein, wherein a proximal end portion of the cartridge is configured to extend at least partially within the distal end portion of the brake tower along the longitudinal axis;
a piston rod coaxially positioned within the brake tower and capable of axial travel within the brake tower and the cartridge along the longitudinal axis; and
a biasing component positioned between the proximal end portion of the cartridge and the distal end portion of the brake tower, wherein the biasing component surrounds a portion of the piston rod.

2. The medical injection device of claim 1, wherein the biasing component is configured to engage an outer surface of the piston rod.

3. The medical injection device of claim 1, wherein the biasing component comprises a plurality of inwardly-projecting protrusions.

4. The medical injection device of claim 3, wherein the plurality of inwardly-projecting protrusions of the biasing component are configured to engage an outer surface of the piston rod.

5. The medical injection device of claim 4, wherein the plurality of inwardly-projecting protrusions are configured to engage the outer surface of the piston rod when the biasing component is in a substantially uncompressed configuration, and further wherein the plurality of inwardly projecting protrusions are configured to disengage from the outer surface of the piston rod when the biasing component is in a substantially compressed configuration.

6. The medical injection device of claim 3, wherein each of the inwardly-projecting protrusions includes a prong configured to engage the piston rod.

7. The medical injection device of claim 3, wherein the plurality of inwardly-projecting protrusions comprises four protrusions.

8. The medical injection device of claim 1, wherein the biasing component comprises a plurality of notches formed therein, wherein the plurality of notches are configured to allow for flexing of engagement surfaces of the biasing component.

9. The medical injection device of claim 8, wherein the biasing component is configured as a leaf spring.

10. The medical injection device of claim 1, wherein the biasing component is formed from metal.

11. The medical injection device of claim 1, wherein the biasing component is formed from polymer.

12. A medical injection device comprising:
a housing having a distal end and a proximal end;
a brake tower having a proximal end portion, a distal end portion, and a sidewall therebetween defining a longitudinal axis;
a cartridge received within the housing distally of the brake tower and holding a composition therein, wherein a proximal end portion of the cartridge is configured to extend at least partially within the distal end portion of the brake tower along the longitudinal axis;
a cartridge housing received within the housing distally of the brake member and configured to hold the cartridge therein;
an injection needle at a distal end of the housing and in fluid communication with the cartridge;
an actuation member at a proximal end of the housing, the actuation member configured to actuate the medical injection device to deliver the composition through the injection needle;
a piston rod coaxially positioned within the brake tower and capable of axial travel within the brake tower and the cartridge along the longitudinal axis; and
a biasing component positioned between the proximal end portion of the cartridge and the distal end portion of the brake tower, wherein the biasing component comprises a plurality of inwardly-projecting protrusions configured to engage an outer surface of the piston rod.

13. The medical injection device of claim 12, wherein the plurality of inwardly-projecting protrusions are configured to engage the outer surface of the piston rod when the biasing component is in a substantially uncompressed configuration, and further wherein the plurality of inwardly projecting protrusions are configured to disengage from the outer surface of the piston rod when the biasing component is in a substantially compressed configuration.

14. The medical injection device of claim 12, wherein each of the inwardly-projecting protrusions includes a prong configured to engage the outer surface of the piston rod.

15. The medical injection device of claim 12, wherein the plurality of inwardly-projecting protrusions comprises four protrusions.

16. The medical injection device of claim 12, wherein the biasing component is formed from metal.

17. The medical injection device of claim 12, wherein the biasing component is formed from polymer.

18. A medical injection device comprising:
a housing having a distal end and a proximal end;
a brake tower having a proximal end portion, a distal end portion, and a sidewall therebetween defining a longitudinal axis;
a cartridge received within the housing distally of the brake tower and holding a composition therein, wherein a proximal end portion of the cartridge is configured to extend at least partially within the distal end portion of the brake tower along the longitudinal axis;
a cartridge housing received within the housing distally of the brake member and configured to hold the cartridge therein;
an injection needle at a distal end of the housing and in fluid communication with the cartridge;
an actuation member at a proximal end of the housing, the actuation member configured to actuate the medical injection device to deliver the composition through the injection needle;
a piston rod coaxially positioned within the brake tower and capable of axial travel within the brake tower and the cartridge along the longitudinal axis; and
a biasing component positioned between the proximal end portion of the cartridge and the distal end portion of the brake tower, wherein the biasing component comprises a partial or full ring member configured to allow for flexing of engagement surfaces of the biasing component.

19. The medical injection device of claim 18, wherein the ring member comprises a plurality of notches which are configured to allow for flexing of the engagement surfaces of the biasing component.

20. The medical injection device of claim 18, wherein the biasing component is configured as at least one of a star washer, Belleville spring, and/or an E-clip.

21. The medical injection device of claim 18, wherein the biasing component is configured to engage an outer surface of the piston rod.
